(19) United States
(12) Patent Application Publication
(10) Pub. No.: US 2021/0350916 A1
(43) Pub. Date: Nov. 11, 2021

(54) Medical Equipment Distribution Assembly

(71) Applicant: Steven Simmons, Rochester, MI (US)

(72) Inventor: Steven Simmons, Rochester, MI (US)

(21) Appl. No.: 17/314,033

(22) Filed: May 6, 2021

Related U.S. Application Data
(60) Provisional application No. 63/020,572, filed on May 6, 2020.

(51) Int. Cl. G16H 40/40; G16H 40/63; G06K 9/00; A61L 2/00

(52) U.S. Cl. CPC G16H 40/40; A61L 2/0047; G06K 9/00228; G16H 40/63

(57) ABSTRACT

A medical equipment distribution assembly includes a housing, an electronic control unit (ECU), a camera assembly, a thermal imaging unit, and/or at least one medical kit. The ECU may be electrically connected with the camera assembly and/or the thermal imaging unit. The ECU may be configured to receive identifying information via the camera assembly and/or the thermal imaging unit. The ECU may be configured to identify an individual substantially proximate the housing and/or the camera assembly; determine if said individual is experiencing a fever; and/or distribute a medical kit to said individual.

### CROSS-REFERENCE

This application claims the benefit of Provisional Application Number 63020572 filed May 6, 2020.

### TECHNICAL FIELD

The present disclosure generally relates to medical equipment distribution assemblies including medical equipment distribution assemblies that may be used in connection with automatically distributing medical supplies including medical equipment, medical kits, and/or personal protective equipment.

### BACKGROUND

This background description is set forth below for the purpose of providing context only. Therefore, any aspect of this background description, to the extent that it does not otherwise qualify as prior art, is neither expressly nor impliedly admitted as prior art against the instant disclosure.

Some medical equipment distribution assemblies may not monitor the use or distribution of medical equipment. For example, some medical equipment distribution assemblies may not be configured to connect with a camera assembly to monitor distribution of medical equipment, and/or assess if an individual is a health risk and should be wearing personal protective equipment.

There is a desire for solutions/options that minimize or eliminate one or more challenges or shortcomings of medical equipment distribution assemblies. The foregoing discussion is intended only to illustrate examples of the present field and is not a disavowal of scope.

### SUMMARY

In embodiments, a medical equipment distribution assembly may include a housing, an electronic control unit (ECU), a camera assembly, a thermal imaging unit, and/or at least one medical kit. The ECU may be electrically connected with the camera assembly and/or the thermal imaging unit. The ECU may be configured to receive identifying information via the camera assembly and/or the thermal imaging unit. The ECU may be configured to identify an individual proximate the housing and/or the camera assembly; determine if said individual is experiencing a fever; and/or distribute a medical kit to said individual.

The foregoing and other potential aspects, features, details, utilities, and/or advantages of examples/embodiments of the present disclosure will be apparent from reading the following description, and from reviewing the accompanying drawings.

### BRIEF DESCRIPTION OF THE DRAWINGS

While the claims are not limited to a specific illustration, an appreciation of various aspects may be gained through a discussion of various examples. The drawings are not necessarily to scale, and certain features may be exaggerated or hidden to better illustrate and explain an innovative aspect of an example. Further, the exemplary illustrations described herein are not exhaustive or otherwise limiting, and are not restricted to the precise form and configuration shown in the drawings or disclosed in the following detailed description. Exemplary illustrations are described in detail by referring to the drawings as follows:

FIG. 1 is a front-perspective view generally illustrating an embodiment of a medical equipment distribution assembly according to teachings of the present disclosure.

FIG. 2 is a rear-perspective view generally illustrating portions of an embodiment of a medical equipment distribution assembly according to teachings of the present disclosure.

FIG. 2A is a front view generally illustrating an embodiment of a medical equipment distribution assembly having a mobile assembly according to teachings of the present disclosure.

FIG. 2B is a front view generally illustrating an embodiment of a medical equipment distribution assembly having a mobile assembly according to teachings of the present disclosure.

FIG. 2C is a front view generally illustrating an embodiment of a medical equipment distribution assembly according to teachings of the present disclosure.

FIG. 3 is a perspective view generally illustrating portions of an embodiment of a medical equipment distribution assembly according to teachings of the present disclosure.

FIG. 4 is a perspective view generally illustrating portions of an embodiment of a medical equipment distribution assembly according to teachings of the present disclosure.

FIG. 5 is a diagram generally illustrating portions of an embodiment of a medical equipment distribution assembly according to teachings of the present disclosure.

FIG. 6 is a perspective view generally illustrating portions of an embodiment of a medical kit according to teachings of the present disclosure.

FIG. 7 is a perspective view generally illustrating portions of an embodiment of a medical kit according to teachings of the present disclosure.

FIG. 8 is a perspective view generally illustrating portions of an embodiment of one or more medical kits according to teachings of the present disclosure.

FIG. 9 is a diagram generally illustrating portions of an embodiment of a medical equipment distribution assembly according to teachings of the present disclosure.

FIG. 10 is a flowchart generally illustrating a method of operating portions of an embodiment of a medical equipment distribution assembly according to teachings of the present disclosure.

### DETAILED DESCRIPTION

Reference will now be made in detail to embodiments of the present disclosure, examples of which are described herein and illustrated in the accompanying drawings. While the present disclosure will be described in conjunction with embodiments and/or examples, it will be understood that they do not limit the present disclosure to these embodiments and/or examples. On the contrary, the present disclosure covers alternatives, modifications, and equivalents.

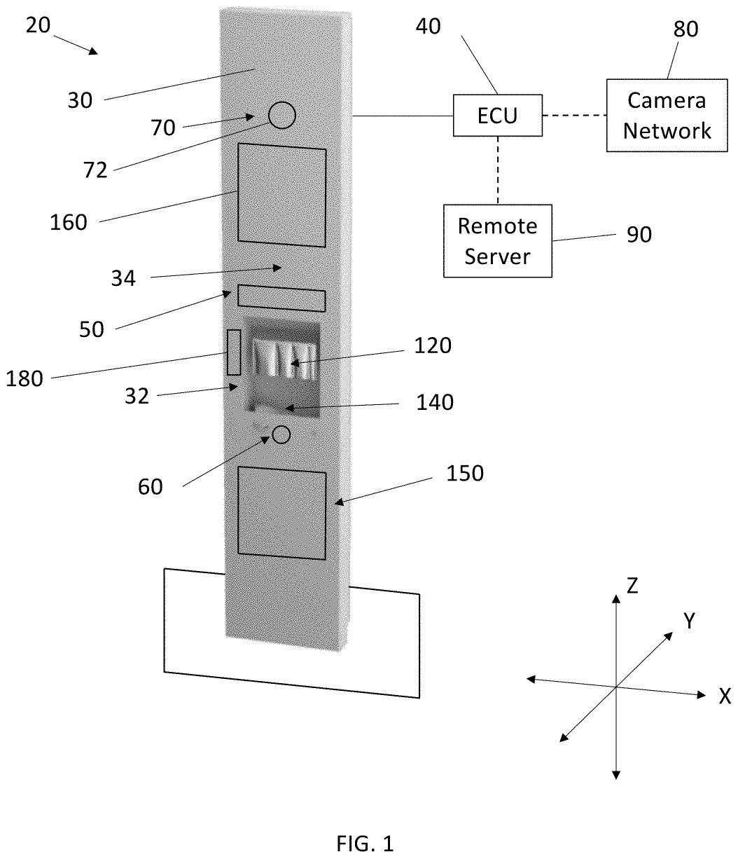

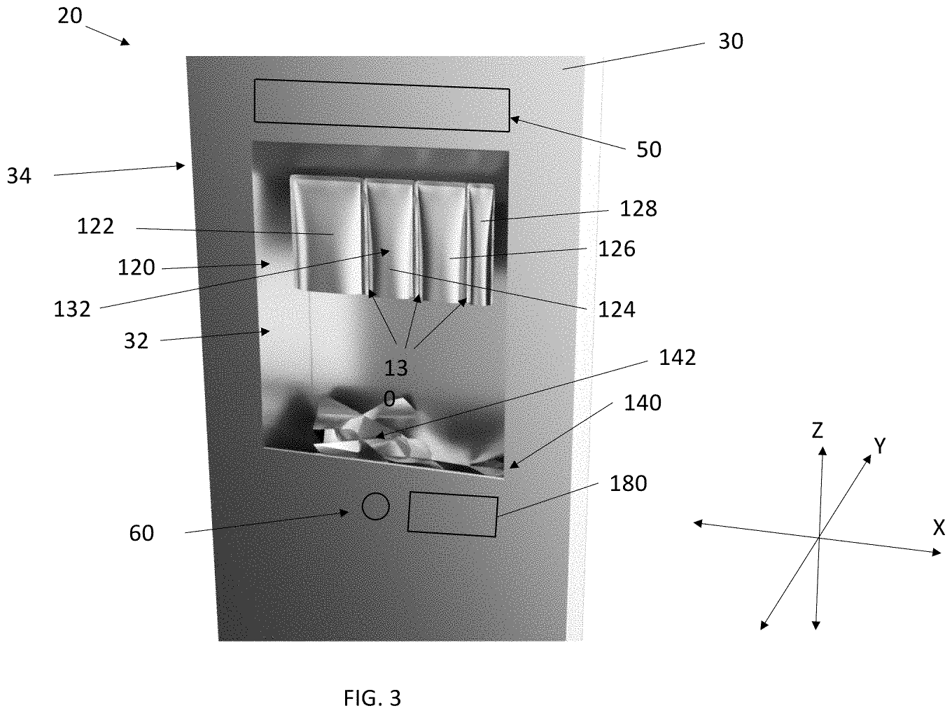

In embodiments, such as generally illustrated in FIGS. 1, 2, 3, 4, and 5, the medical equipment distribution assembly 20 may include a housing 30, an electronic control unit (ECU) 40, a sensor assembly 60, a camera assembly 70, and/or a thermal imaging unit 110. The medical equipment distribution assembly 20 may include one or more of a variety of medical kits 120. The ECU 40 may be connected with the housing 30, the camera assembly 70, and/or the thermal imaging unit 110 to automatically monitor individuals (e.g., for one or more of a variety of medical conditions) substantially proximate the medical equipment distribution assembly 20 (e.g., the housing 30), and/or to provide one or more medical kits 120 to said individuals. The medical equipment distribution assembly 20 may be configured to (i) determine if an individual is exhibiting health irregularities (e.g., a fever); (ii) determine if said individual is using (e.g., wearing) medical equipment; and/or (iii) provide medical equipment (e.g., via a medical kit) for said individual.

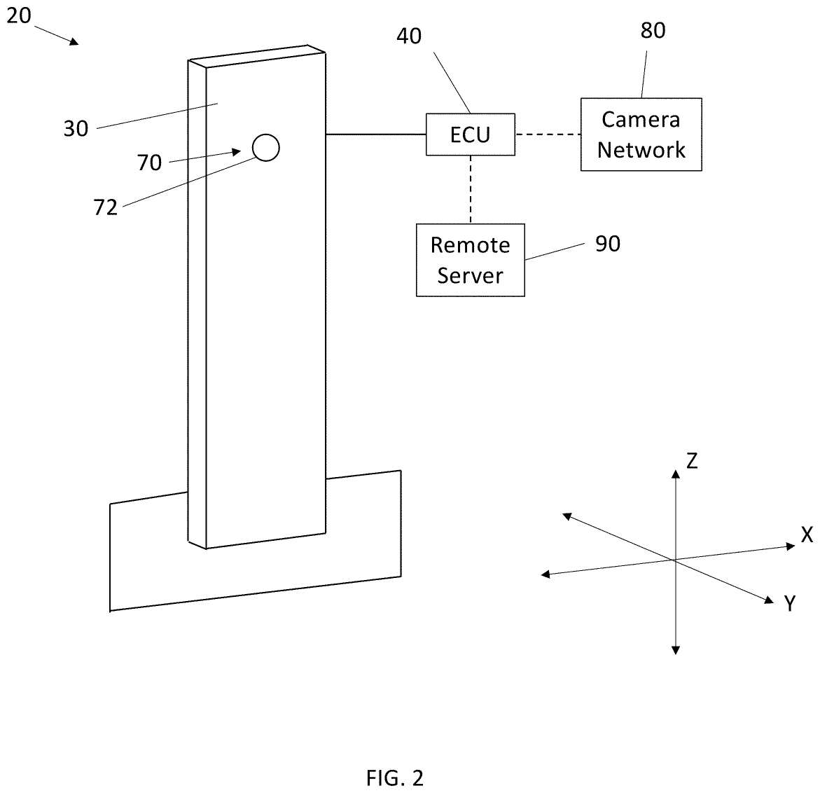

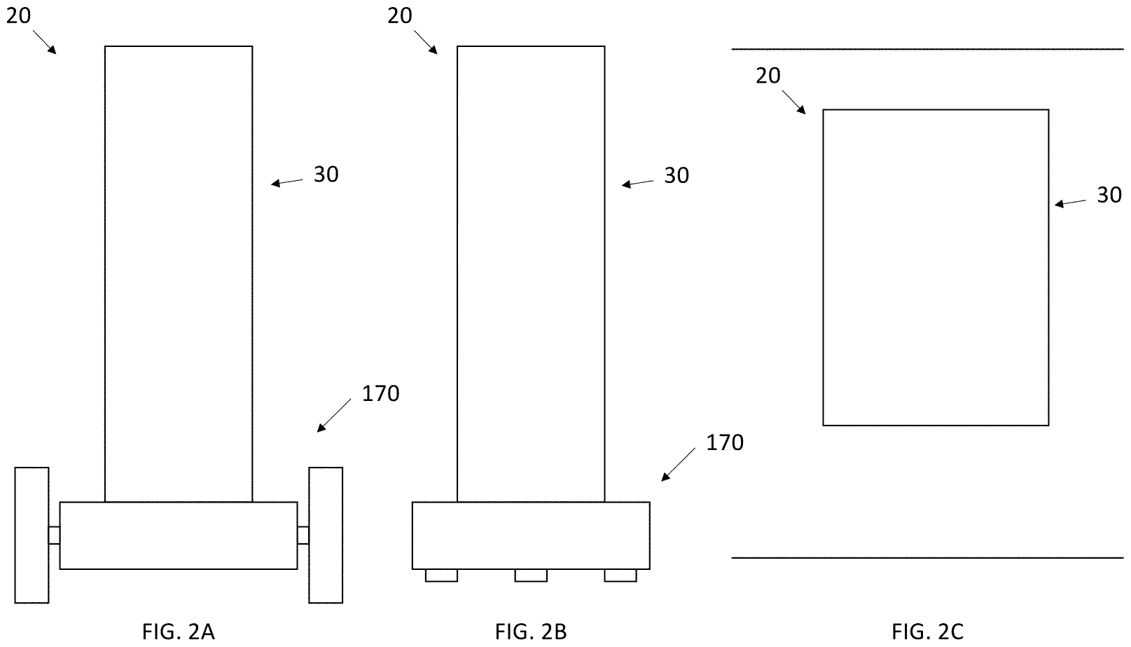

With embodiments, such as generally illustrated in FIGS. 1 and 2, the medical equipment distribution assembly 20 may include a housing 30. The housing 30 may include one or more of a variety of shapes, sizes, and/or configurations. For example and without limitation, the housing 30 may extend substantially in the Z-direction (e.g., vertically) and/or the housing 30 may be generally rectangular. The housing 30 may include one or more of a variety of materials. For example and without limitation, the housing 30 may include one or more of a variety of polymers, steel, copper, silver, and/or wood (e.g., a coating/plating of steel, copper, silver, and/or wood). In embodiments, the ECU 40, the camera assembly 70, the thermal imaging unit 110, and/or the sensor assembly 60 may be disposed at least partially within the housing 30. Additionally or alternatively, the camera assembly 70 and/or the thermal imaging assembly 110 may be disposed outside the housing 30 (e.g., the ECU 40 may be wirelessly connected with the camera assembly 70 and/or the thermal imaging unit 110).

With embodiments, the housing 30 may be configured to store and/or distribute (e.g., automatically and/or selectively) one or more of a variety of medical kits 120. The housing 30 may include an aperture 32 and/or the medical equipment distribution assembly 20 may be configured to distribute medical kits via the aperture 32. The housing 30 may be one or more of a variety of sizes such that various quantities of medical kits 120 may be disposed within the housing 30. For example and without limitation, larger housings may correspond to a larger quantity of medical kit storage capacity, and/or smaller housings may correspond to a smaller quantity of medical kit storage capacity.

In embodiments, the housing 30 may be connected with a mobile assembly 170. The mobile assembly 170 may be configured to move/transport the medical equipment distribution assembly 20 to one or more of a variety of positions. The mobile assembly 170 may be disposed substantially proximate a bottom portion of the housing 30. For example and without limitation, the mobile assembly 170 may include any number of actuators, motors, and/or wheels. The mobile assembly 170 may include a plurality of wheels that may be configured to move the housing over one or more of a variety of surfaces. For example and without limitation, such as generally illustrated in FIG. 2A, the medical equipment distribution assembly 20 may include a mobile assembly 170 configured to traverse uneven terrain (e.g., surfaces outside and/or found in construction sites that may lack traction and/or may not be substantially planar/flat). The mobile assembly 170 may include military-grade wheels such that the medical equipment distribution assembly 20 may traverse a variety of debris without preventing and/or substantially limiting movement of the housing 30. Additionally or alternatively, such as generally illustrated in FIG. 2B, the mobile assembly 170 may include a plurality of wheels that may be configured to transport the housing over indoor surfaces (e.g., substantially flat surfaces and/or non-slip surfaces). Mobile assemblies 170 configured for indoor use may include wheels having a smaller diameter and/or motors having lower torque than that of mobile assemblies 170 configured for outdoor surfaces. With embodiments, such as generally illustrated in FIG. 2C, the housing 30 may not include a mobile assembly 170 and/or may be configured to be connected to a wall (e.g., the housing may be mounted and/or positioned on a wall for fixed operation).

In embodiments, such as generally illustrated in FIGS. 1, 3, and 5, the housing may include an sanitization portion 50. The sanitization portion 50 may be electrically connected (e.g., via a wired and/or wireless connection) with the ECU 40 such that the ECU 40 may control operation of the sanitization portion 50. The sanitization portion 50 may be configured to at least partially sanitize and/or disinfect the medical kits 120 (e.g., via chemicals and/or light). For example and without limitation, the sanitization portion 50 may be configured to emit/transmit ultra violet (UV) light (e.g., emitted light may include UVC light). The sanitization portion 50 may be disposed substantially proximate the aperture 32 such that the sanitization portion 50 may sanitize/disinfect the one or more medical kits 120 prior to distribution. With embodiments, the sanitization portion 50 may be disposed at least partially within the housing 30 to periodically sanitize/disinfect the medical kits 120 stored within the housing 30 (e.g., which may be controlled by the ECU 40). Periodically sanitizing/disinfecting one or more medical kits 120 disposed within the housing 30 may reduce the likelihood of compromising medical kits 120 (e.g., exposing medical kits to germs, virus, and/or disease). The ECU 40 may be configured to conduct and/or verify periodic sanitization of the medical kits 120 and/or the ECU 40 may provide an indication (e.g., audible and/or visual) for completed periodic sanitization and/or incomplete periodic sanitization. For example and without limitation, if the ECU 40 is not able to validate sanitization of one or more medical kits 120 via the sanitization portion 50, the medical equipment distribution assembly 20 may not distribute medical equipment (e.g., medical kits 120). The ECU 40 may be configured to close/seal the aperture 32 such that medical kits 120 may not be accessed and/or provided. The ECU 40 may transmit a notification of incomplete sanitization to a remote server 90, such that the medical equipment distribution assembly 20 may be replaced and/or repaired.

With embodiments, such as generally illustrated in FIGS. 1, 2, and 5, the medical equipment distribution assembly 20 may include a sensor assembly 60. The sensor assembly 60 may be disposed substantially proximate the aperture 32. The sensor assembly 60 may be electrically connected (e.g., wired and/or wirelessly) with the ECU 40. The sensor assembly 60 may include one or more of a variety of sensors. For example and without limitation, the sensor assembly 60 may include one or more proximity sensors, motion sensors, temperature sensors, and/or cameras. The sensor assembly 60 may be configured to receive proximity input and/or the ECU 40 may determine when an individual is substantially proximate the medical equipment distribution assembly 20 such as to automatically and/or selectively distribute a medical kit to said individual. One or more of a variety of inputs may be received by the sensor assembly 60 such as temperature change, motion detection, and/or facial recognition. The sensor assembly 60 may be configured to receive and/or process retinal information of nearby individuals that may indicate vital information (e.g., oxygen saturation). The sensor assembly 60 may be disposed substantially proximate a first side 34 of the housing 30 and/or the aperture 32 such as to receive input when an individual is substantially proximate to the medical equipment distribution assembly 20. Upon receiving input via the sensor assembly 60, the medical equipment distribution assembly 20 may selectively distribute one or more of a variety of medical kits 120. For example and without limitation, as an individual approaches the medical equipment distribution assembly 20, the ECU 40 may receive information via the sensor assembly 60 such that the medical equipment distribution assembly 20 may distribute/provide a medical kit 120 to the identified individual. Medical kits 120 may be mechanically distributed from the housing 30 (e.g., such that an individual may pull/tear a medical kit 120 to remove and/or use); and/or the medical kits 120 may be electronically distributed from the housing 30 (e.g., such that a medical kit may move/drop from the housing into the individual's hands).

In embodiments, such as generally illustrated in FIGS. 1, 2, and 5, the medical equipment distribution assembly 20 may include a camera assembly 70 that may be configured to identify individuals. The ECU 40 may be connected (e.g., wired and/or wirelessly) with the camera assembly 70 such that the ECU 40 may receive identification information via the camera assembly 70. The camera assembly 70 may include any number of cameras connected to the housing 30 in one or more of a variety of positions. For example and without limitation, the camera assembly 70 may include a first camera 72 that may be disposed on the first side 32 of the housing, and/or the camera assembly 70 may include a second camera 74 that may be disposed on a second side 34 of the housing. The first side 32 of the housing 30 may be opposite the second side 34 of the housing 30. The first camera 72 may collect identification information for individuals walking towards the first side 32, and/or the second camera 74 may collect identification information for individuals walking towards the second side 34. The camera assembly 70 may include any number of cameras disposed on and/or in the housing 30 such that identification information may be collected for an individual in one or more of a variety of positions relative to the medical equipment distribution assembly 20. The medical equipment distribution assembly 20 may be configured to distribute medical kits 120 from the first side 32 and/or the second side 34 of the housing 30.

With embodiments, such as generally illustrated in FIGS. 1, 2, and 5, the ECU 40 may be connected with a camera network 80. The camera network 80 may include one or more cameras that may be wirelessly connected (and/or via a wired connection) with the ECU 40. The camera network 80 may include one or more cameras disposed outside of the housing 30, such as cameras installed on walls and/or ceilings of a structure. Additionally or alternatively, one or more cameras of the camera network 80 may be disposed in appropriate locations such as to capture identification information without interference (e.g., interference from objects, individuals, and/or structures that may impede camera views of individuals). Appropriate locations may include locations in which the cameras of the camera network 80 are disposed substantially proximate a ceiling and/or on a wall substantially above heads of individuals. In embodiments, the ECU 40 may be configured to connect and/or communicate with existing security systems (e.g., security cameras and/or surveillance equipment).

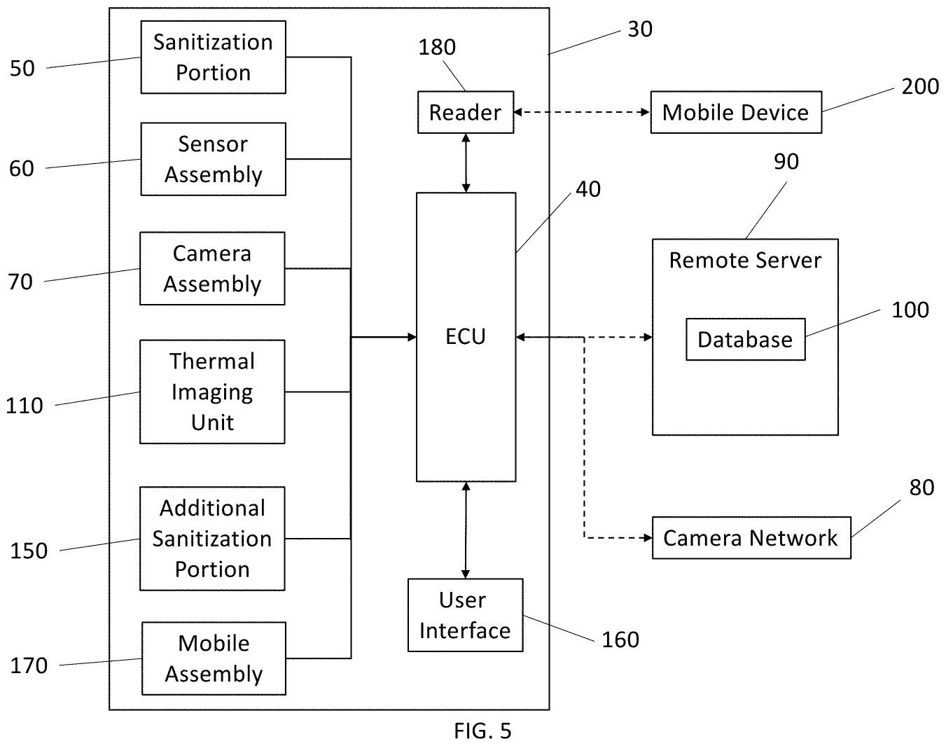

In embodiments, such as generally illustrated in FIG. 5, the ECU 40 may be configured to communicate with the camera assembly 70 and/or the camera network 80, which may include a combination of cameras disposed within the housing 30 and/or outside the housing 30. The ECU 40 may receive identification information via the camera assembly 70 and/or the camera network 80 to facilitate facial recognition/identification of individuals substantially proximate the medical equipment distribution assembly 20 (e.g., individuals walking by the housing 30 from one or more of a variety of directions). The medical equipment distribution assembly 20 may include and/or be connected with a remote server 90. The remote server 90 may be wirelessly connected with the ECU 40 such that the ECU 40 may transfer information to the remote server 90 and/or the ECU 40 may receive information via the remote server 90. The remote server 90 may be connected with one or more of a variety of electronic devices (e.g., phones, tablets, processing units, and/or computer networks). Additionally or alternatively, the remote server 90 may include and/or be connected with a database 100. The ECU 40 may be configured to upload information received by the sensor assembly 60, the camera assembly 70, and/or the camera network 80 to the remote server 90 and/or database 100. The database 100 may receive information and/or may store information corresponding to the identities of individuals detected by the camera assembly 70 and/or the camera network 80. The database 100 may be configured to generate profiles corresponding with the identified individuals to monitor activity via the medical equipment distribution assembly 20. The profiles may include information regarding (i) the location of the identified individual, (ii) the time at which the identified individual was substantially proximate the housing 30, (iii) employee information (e.g., to verify authorized access to one or more areas), (iv) if the individual demonstrated wearing medical equipment (e.g., personal protective equipment), and/or (v) the medical state of the identified individual.

With embodiments, the medical equipment distribution assembly 20 may be configured to determine if an individual is using appropriate/proper medical equipment, and/or if an individual received a medical kit 120. The ECU 40 may communicate with the sensor assembly 60, the camera assembly 70, and/or the camera network 80 to identify individuals without medical equipment. The ECU 40 may be configured to communicate with the remote server 90 to transmit a notification (e.g., audibly and/or visually) that an individual does not have appropriate medical equipment. The notification may provide time, location, identity, and/or occupational information for the identified individual without appropriate medical equipment.

In embodiments, such as generally illustrated in FIG. 5, the medical equipment distribution assembly 20 may include a thermal imaging unit 110. The thermal imaging unit 110 may be connected with the ECU 40, the camera assembly 70, the camera network 80, and/or the remote server 90. The thermal imagining unit 110 may be a portion of the camera assembly 70. For example and without limitation, the cameras of the camera assembly 70 and/or the camera network 80 may include thermal imaging capabilities (e.g., the cameras may be thermal imaging cameras). The thermal imaging unit 110 may be configured to sense/determine temperatures of individuals substantially proximate the housing 30, and/or cameras of the camera assembly 70 and/or the camera network 80. The ECU 40 may be configured to determine and/or transmit medical information of an individual to the remote server 90 and/or database 100. For example and without limitation, medical information may include body temperature. With embodiments, if the ECU 40 determines that a body temperature of an individual is greater than a first threshold (e.g., about 98.6 degrees Fahrenheit or more or less), the ECU 40 may transmit a first notification and/or may flag the individual's profile within the remote server 90 and/or database 100. If the ECU 40 determines that the body temperature of the individual is greater than a second threshold (e.g., about 100.4 degrees Fahrenheit or more or less), the ECU 40 may transmit a second notification and/or may flag the individual's profile. The ECU 40 may be configured transmit the first notification and/or the second notification to the remote server 90 (e.g., which may be connected to one or more of a variety of computing devices and/or security systems). An individual may obtain a medical kit 120 if said individual does not exhibit symptoms of illness. One or more of a variety of payment systems may be connected with the ECU 40 and/or the housing 30 such that individuals may purchase medical kits 120 (e.g., personal protective equipment). The ECU 40 may be connected with one or more electronic devices (e.g., via a remote network, Bluetooth, and/or any variety of wireless communication) such that the individual may purchase medical kits 120 without contacting the housing 30. Additionally or alternatively, the ECU 40 may connect with information input devices (e.g., Bluetooth keyboards) for employees to enter information/data and/or to perform maintenance.

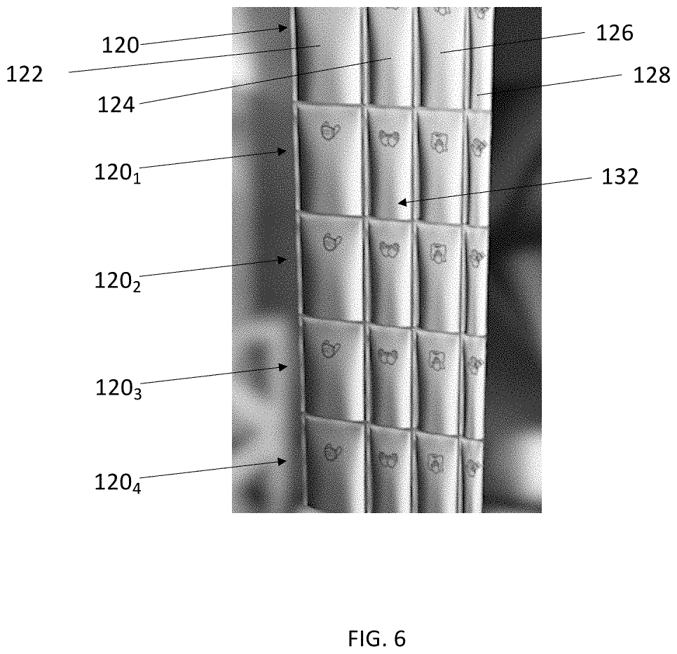

With embodiments, such as generally illustrated in FIGS. 1, 3, 4, 6, and 7, the medical equipment distribution assembly 20 may be configured to provide one or more medical kits 120 to individuals sensed/monitored/recorded by the ECU 40 (e.g., via the sensor assembly 60, the camera assembly 70, and/or the camera network 80). The medical kit 120 may include one or more of a variety of personal protective equipment (e.g., medical equipment). For example and without limitation, the medical kit 120 may include a first portion 122, a second portion 124, a third portion 126, and/or a fourth portion 128. The first portion 122, the second portion 124, the third portion 126, and/or the fourth portion 128 may protect contents of the medical kit 120 (e.g., personal protective equipment) from exposure to diseases, viruses, and/or germs. The portions 122, 124, 126, 128 may be configured as cartridges and/or packets to protect and/or distribute medical equipment. The first portion 122 may include a mask, the second portion 124 may include protective gloves (e.g., nitrate, latex, and/or latex-free gloves), the third portion 126 may include a disinfectant wipe, and/or the fourth portion 128 may include disinfectant liquid, gel, and/or aerosol. The portions 122, 124, 126, 128 of the medical kit 120 may be separately sealed such that opening and/or use of the first portion 122 does not compromise (e.g., expose the medical equipment) the second portion 124, the third portion 126, and/or the fourth portion 128. For example and without limitation, the portions 122, 124, 126, 128 may include perforated portions 130 such that portions 122, 124, 126, 128 may be easily separated and opened. Opening the second portion 124 may not compromise medical equipment disposed in the first portion 122, the third portion 126, and/or the fourth portion 128. Opening the third portion 126 may not compromise medical equipment disposed in the first portion 122, the second portion 124, and/or the fourth portion 128. Opening the fourth portion 128 may not compromise medical equipment disposed in the first portion 122, the second portion 124, and/or the third portion 126. One or more medical kits 120_{N} may be at least partially connected (e.g., such as generally illustrated in FIG. 6) such that a tearing and/or ripping motion may separate the medical kits 120 as the housing distributes the medical kits 120_{N}.

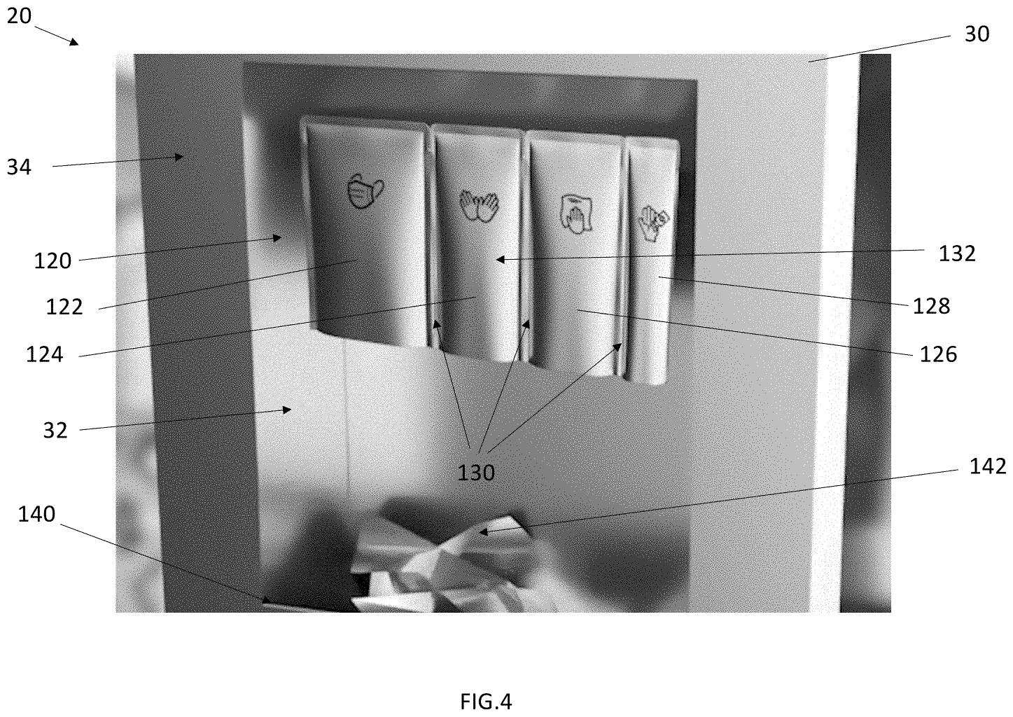

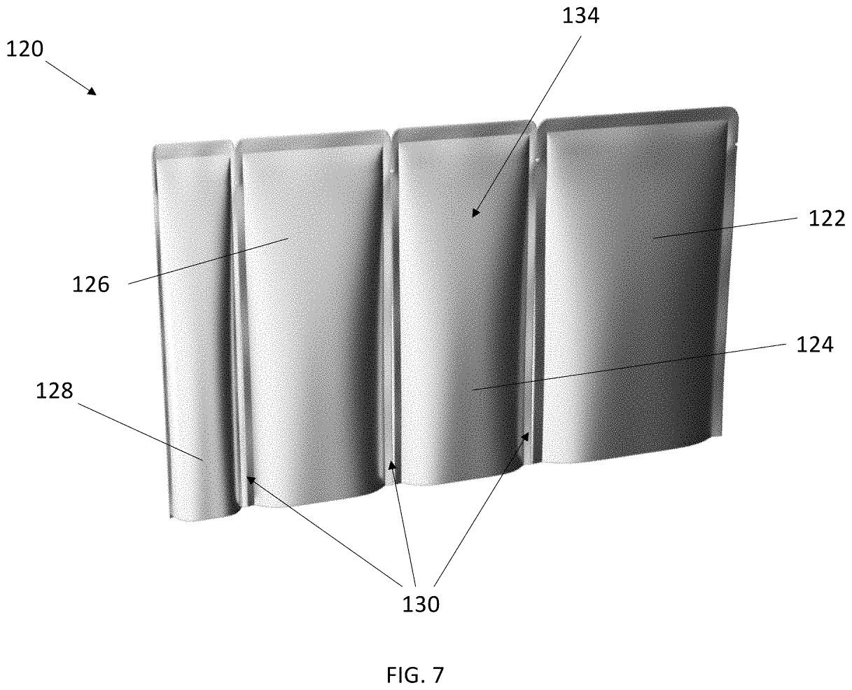

In embodiments, the medical kit portions 122, 124, 126, 128 may include one or more of a variety of materials and/or may include one or more of variety of shapes, sizes, and/or configurations. For example and without limitation, the medical kit portions 122, 124, 126, 128 may include disposable/biodegradable materials. The medical kit portions may include a first side 132 and/or a second side 134. The first side 132 may be opposite the second side 134. As generally illustrated in FIG. 4, the first side 132 of the medical kit portions 122, 124, 126, 128 may include a copper coating and/or foil (e.g., biodegradable foil). Such as generally illustrated in FIG. 7, the second side 134 of the medical kit portions 122, 124, 126, 128 may include a transparent material (e.g., a polymer) such that contents of the medical kit 120 may be viewed from the second side 134 (e.g., for security inspection). The sanitization portion 50 may be configured to emit UV light through the second side 134 of the medical kit 120. UV light may pass through the second side 134 to sanitize contents of the first portion 122, the second portion 124, the third portion 126, and/or the fourth portion 128 of the medical kit 120. The portions 122, 124, 126, 128 may include a thickness and/or chemical composition such that UVC light (e.g., including a wavelength of about 100 to 290 nm) enters to disinfect the contents. The portions 122, 124, 126, 128 of the medical kit 120 may include a material having flexible characteristics and/or UVC light penetrating material (e.g., acrylics, polymers, plastics, glass, etc.). The sanitization portion 50 may be configured to sanitize the outer surface of the medical kit 120 and/or contents disposed at least partially within the portions 122, 124, 126, 128. Additionally or alternatively, additional sanitization portions disposed outside the housing 30 may be configured to sanitize the one or more medical kits 120 prior to loading (e.g., when loading the medical equipment distribution unit with medical kits 120).

With embodiments, the medical kit portions 122, 124, 126, 128 may include medical equipment (e.g., personal protective equipment) for one or more of a variety of locations. For example and without limitation, medical kit portions 122, 124, 126, 128 may include equipment configured for use with construction sites (e.g., protective eyewear and/or other medical equipment used on construction sites). Additionally or alternatively, the medical kit portions 122, 124, 126, 128 may include equipment configured for use with a hospital (e.g., gowns, shoe covers, face shields, etc.) a casino, and/or a concert venue.

In embodiments, such as generally illustrated in FIGS. 1, 3, and 4, the medical equipment distribution assembly may include a recess 140. The recess 140 may be configured as a receptacle for used medical kit portions 142. The housing 30 may include an additional sanitization portion 150. The additional sanitization portion 150 may be configured to disinfect used medical kit portions 142 disposed in the recess 140. The additional sanitization portion 150 may disinfect the used medical kit portions 142 via UV light and/or application of disinfecting chemicals.

With embodiments, such as generally illustrated in FIG. 1, the medical equipment distribution assembly may include a user interface and/or display 160. The user interface 160 may be disposed substantially towards an upper portion of the housing 30 (e.g., such as to be disposed substantially proximate the head of an individual). The user interface 160 may be configured to receive input from an individual in one or more of a variety of manners. For example and without limitation, the user interface 160 may receive information from one or more individuals via audible commands and/or physical interactions (e.g., such as via a touch-screen display). The user interface 160 may be electrically connected with the ECU 40, the sensor assembly 60, the sanitization portion 50, the camera network 80, the remote server 90, the thermal imaging unit 110 and/or the additional sanitization portion 150. For example and without limitation, additional medical equipment may be requested by an individual via the user interface 160 and/or an individual may supply medial information via the user interface 160 (e.g., medical history and/or information regarding the current medical state of the individual).

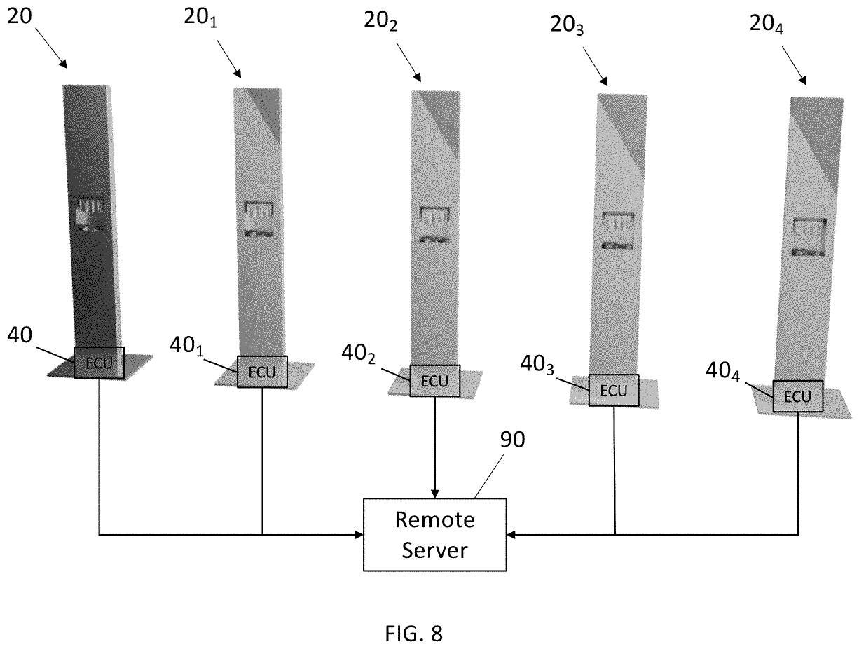

In embodiments, such as generally illustrated in FIG. 8, one or more medical equipment distribution assemblies 20_{N} may be configured to communicate with other medical equipment distribution assemblies. The medical distribution assembly 20 may be configured to communicate with any number of additional medical distribution assemblies 20₁, 20₂, 20₃, 20₄ (e.g., via wired and/or wireless connection) to provide medical kits 120 to individuals. Additional medical equipment distribution assemblies may be connected to accommodate high traffic areas.

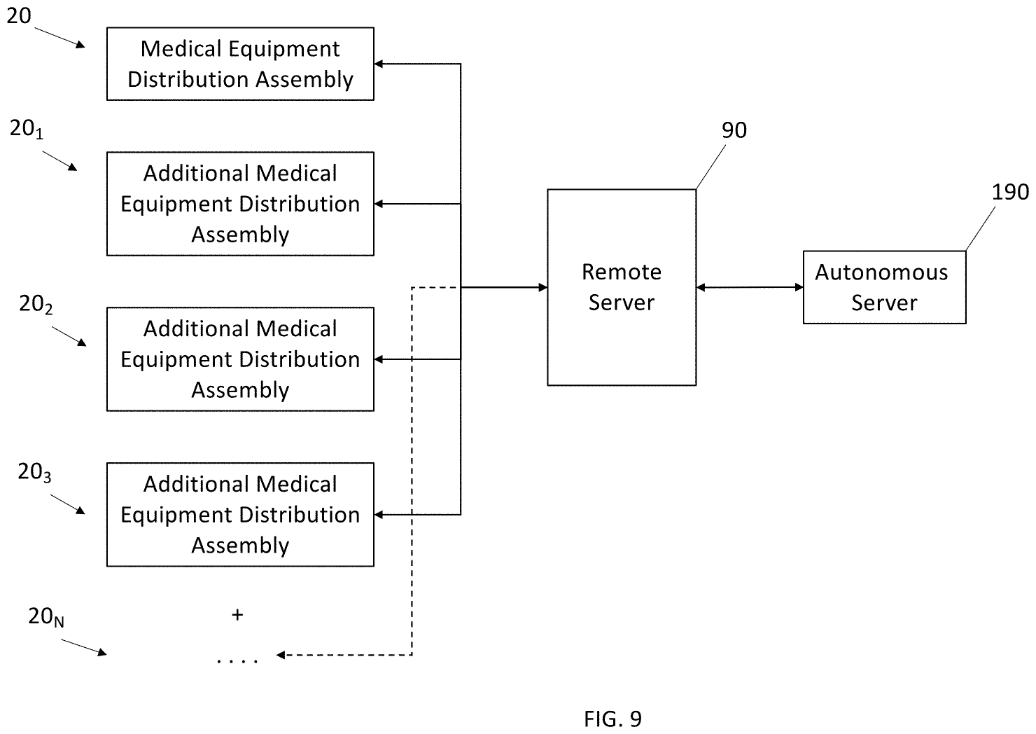

With embodiments, such as generally illustrated in FIG. 9, the remote server 90 may include and/or be connected with an autonomous server 190. Functions of the remote server 90 and/or the autonomous server 190 may be combined into a single server and/or any number of servers. The autonomous server 190 may include any number of ECU's and/or processors to control and/or position the medical equipment distribution assemblies 20. For example and without limitation, the medical equipment distribution assemblies 20 (e.g., the one or more ECUs 40) may be configured to receive information (e.g., commands) from the autonomous server 190 and/or the remote server 90. The remote server 90 and/or the autonomous server 190 may be configured to determine one or more of a variety of operational information for the medical equipment distribution assemblies 20. For example and without limitation, the remote server 90 and/or the autonomous server 190 may transmit commands to the medical equipment distribution assemblies 20 to perform one or more of a variety of functions which may include a shutdown command (e.g., such as not to distribute medical kits); a movement command (e.g., such as to reposition the assembly to increase likelihood of equipment distribution); and/or a recall command (e.g., such as to return the assembly 20 to a maintenance station to recharge, resupply with medical kits, and/or for repair). Maintenance stations may operate at least partially autonomously, such as to reduce human interaction with the medical equipment distribution assemblies 20 (e.g., increasing the sanitary nature of the assemblies 20). The maintenance station may be configured to load additional medical kits 120 into the housings 30 such that medical kits 120 are not exposed to germs, virus, and/or disease. Additionally or alternatively, the maintenance stations may be configured to sanitize the one or more medical equipment distribution assemblies 20 and/or the medical kits 120 disposed within the housings 30.

In embodiments, the remote server 90 and/or the autonomous server 190 may be connected with the camera assembly 70 and/or the camera network 80. The remote server 90 and/or the autonomous server 190 may be configured to receive foot-traffic information from the camera network. For example and without limitation, the camera network 80 may monitor foot traffic within a building via one or more security cameras. The camera network 80 may transmit foot traffic information to the remote server 90 and/or the autonomous server 190. The servers may be configured to determine positions (e.g., via GPS, RFID, etc.) of the medical equipment distribution assemblies 20 to best address highly trafficked areas (e.g., entrances to meeting rooms, presentation venues, entrances, exits, etc.). Positioning medical equipment distribution assemblies substantially proximate highly trafficked areas may increase the effectiveness of distributing medical kits 120 to individuals. For example and without limitation, the servers may include event information indicating an event schedule. The autonomous server 190 and/or the remote server 90 may determine quantities and/or positions of medical equipment distribution assemblies 20_{N} in correlation with the event schedule. The autonomous server 190 and/or the remote server 90 may position medical equipment distribution assemblies 20_{N} substantially proximate entrances and/or exits to events prior to the arrival of individuals. The medical equipment distribution assemblies 20 may provide medical kits 120 prior to the start of an event, and/or the medical equipment distribution assemblies 20 may move to a different locations upon commencement of said event.

With embodiments, the medical equipment distribution assembly 20 may include a reader 180 (e.g., a QR code scanner, barcode scanner, etc.). The reader 180 may be disposed on the first side of the housing 30, and/or the reader 180 may be disposed on an outside of the housing 30 such that an individual may access (e.g., transmit and/or receive information/data) the reader upon standing substantially proximate the housing 30. The reader 180 may be configured to receive information from the individual via a mobile device 200 (e.g., see FIG. 5). For example and without limitation, the reader 180 may receive payment information (e.g., via a wireless payment application/interface), identifying information, location information (e.g., identifying previous locations of said individual), provide an automatic download for a contact tracing app (e.g., identifying persons that said individual may have been in close contact with), and/or access information stored on an individual's mobile device 200.

In embodiments, the reader 180 may be configured to recognize and/or identify a specific individual and/or provide a medical kit 120 in correlation with the information received by the reader 180. For example and without limitation, the individual may download an application (e.g., an application synced with the ECU 40, remote network 90, and/or autonomous network 190) upon receiving instructions via the medical equipment distribution assembly 20 (e.g., the user interface). The individual may create a profile via the application and/or may use the application to communicate with the medical equipment distribution assembly 20. For example and without limitation, the ECU 40 may be configured to distribute a medical kit 120 at no cost upon receiving confirmation that said individual downloaded the application. Downloading the application may provide location information of the individual's mobile device 200 to the remote server 90 and/or autonomous server 190 (e.g., which may be used to determine an individual's level of risk for illness and/or disease). Location information may include a listing of previous locations and/or information indicating the number of other individuals that may have been in substantial proximity to said individual. If an individual does not communicate with the medical equipment distribution assembly 20 via the application interface, the medical equipment distribution assembly 20 may charge the individual for a medical kit 120 (e.g., which may be paid via one or more of a variety of wireless payments).

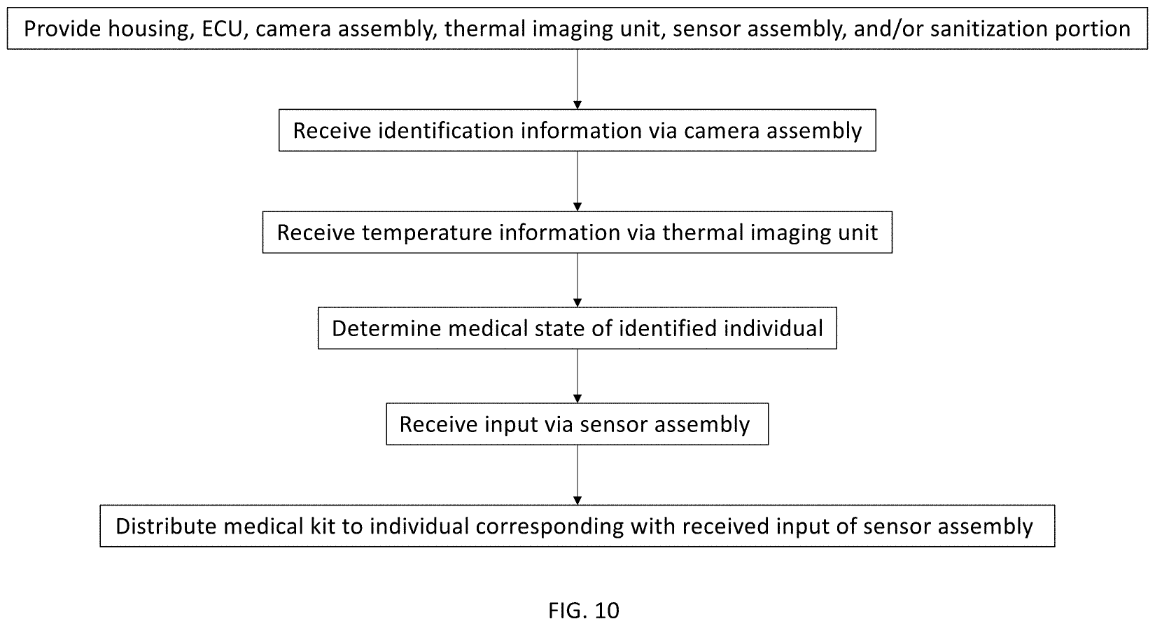

With embodiments, such as generally illustrated in FIG. 10, a method 220 of operating the medical equipment distribution assembly 20 may include providing a housing 30, an ECU 40, a camera assembly 70, a thermal imaging unit 110, a sensor assembly 60, and/or a sanitization portion 50. The ECU 40 may be configured to identify individuals walking substantially proximate the housing 30 and/or the cameras of the camera assembly 70 and/or camera network 80. The method may include the ECU 40 receiving identifying information from the one or more cameras, and/or the comparing the identifying information with information (e.g., employee information, invitee information, etc.) stored on the database 100 via the remote server 90. The method may include comparing the identifying information with information on the database 100 to validate and/or establish the identity of one or more individuals. The method may include determining the medical condition/state of one or more individuals via the thermal imaging unit 110. The ECU 40 may receive body temperature information via the thermal imaging unit 110 to determine if one or more individuals are a health risk (e.g., individuals that may be exhibiting symptoms associated with diseases, viruses, illnesses, etc.). The method may include identifying an individual exhibiting a fever, and/or monitoring whether the individual has received a medical kit 120. Additionally or alternatively, the method may include determining whether the individual is wearing medical equipment (e.g., personal protective equipment) via the camera assembly 70 and/or camera network 80. The method may include transmitting an alert/notification to the remote server 90 (e.g., one or more electronic devices) to indicate an individual that may be compromising the safety of other individuals. If an alert is transmitted, the method may include automatically closing doors and/or isolating portions of a building such as to isolate an individual that may be at risk of disease and/or virus.

In embodiments, the method may include receiving input from an individual via the sensor assembly 60. The sensor assembly 60 may receive motion gestures and/or audible commands to distribute medial kits 120. For example and without limitation, the sensor assembly 60 may receive input to provide a medical kit 120 to an individual. The method may include prompting the individual for additional information (e.g., medical history, current medical symptoms, activities expected to be performed, and/or occupation) to determine the medical equipment required by the individual. The method may include selecting a medical kit 120 to distribute to the individual, and/or sanitizing the medical kit 120 prior to distribution via the sanitization portion 50. The sanitization portion 50 may be automatically activated once a medical kit 120 is selected/queued for the individual. The method may include receiving used (and/or unused) medical kit portions via the recess, and/or sanitizing the used medical kit portions via an additional sanitization unit disposed at least partially within and/or substantially proximate to the recess.

In examples, an ECU 40 may include an electronic controller and/or include an electronic processor, such as a programmable microprocessor and/or microcontroller. The ECU 40 may be configured for machine learning, artificial intelligence (AI) and/or intelligent operations. The ECU 40 may include AI capabilities, such as determining locations for medical equipment distribution assemblies based on received information (e.g., from mobile devices, cameras, servers, and/or applications). For example and without limitation, the ECU 40 may include one or more of a variety of processors, microcontrollers, and/or controllers to effectively analyze and/or process video information (e.g., via the camera network and/or camera assembly), thermal information, facial recognition, and/or foot-traffic flow. In embodiments, an ECU 40 may include, for example, an application specific integrated circuit (ASIC). An ECU 40 may include a central processing unit (CPU), a memory (e.g., a non-transitory computer-readable storage medium), and/or an input/output (I/O) interface. An ECU 40 may be configured to perform various functions, including those described in greater detail herein, with appropriate programming instructions and/or code embodied in software, hardware, and/or other medium. In embodiments, an ECU 40 may include a plurality of controllers. In embodiments, an ECU 40 may be connected to a display, such as a touchscreen display.

Various examples/embodiments are described herein for various apparatuses, systems, and/or methods. Numerous specific details are set forth to provide a thorough understanding of the overall structure, function, manufacture, and use of the examples/embodiments as described in the specification and illustrated in the accompanying drawings. It will be understood by those skilled in the art, however, that the examples/embodiments may be practiced without such specific details. In other instances, well-known operations, components, and elements have not been described in detail so as not to obscure the examples/embodiments described in the specification. Those of ordinary skill in the art will understand that the examples/embodiments described and illustrated herein are non-limiting examples, and thus it can be appreciated that the specific structural and functional details disclosed herein may be representative and do not necessarily limit the scope of the embodiments.

Reference throughout the specification to “examples, “in examples,” “with examples,” “various embodiments,” “with embodiments,” “in embodiments,” or “an embodiment,” or the like, means that a particular feature, structure, or characteristic described in connection with the example/embodiment is included in at least one embodiment. Thus, appearances of the phrases “examples, “in examples,” “with examples,” “in various embodiments,” “with embodiments,” “in embodiments,” or “an embodiment,” or the like, in places throughout the specification are not necessarily all referring to the same embodiment. Furthermore, the particular features, structures, or characteristics may be combined in any suitable manner in one or more examples/embodiments. Thus, the particular features, structures, or characteristics illustrated or described in connection with one embodiment/example may be combined, in whole or in part, with the features, structures, functions, and/or characteristics of one or more other embodiments/examples without limitation given that such combination is not illogical or non-functional. Moreover, many modifications may be made to adapt a particular situation or material to the teachings of the present disclosure without departing from the scope thereof.

It should be understood that references to a single element are not necessarily so limited and may include one or more of such element. Any directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of examples/embodiments.

Joinder references (e.g., attached, coupled, connected, and the like) are to be construed broadly and may include intermediate members between a connection of elements and relative movement between elements. As such, joinder references do not necessarily imply that two elements are directly connected/coupled and in fixed relation to each other. The use of “e.g.” in the specification is to be construed broadly and is used to provide non-limiting examples of embodiments of the disclosure, and the disclosure is not limited to such examples. Uses of “and” and “or” are to be construed broadly (e.g., to be treated as “and/or”). For example and without limitation, uses of “and” do not necessarily require all elements or features listed, and uses of “or” are inclusive unless such a construction would be illogical.

While processes, systems, and methods may be described herein in connection with one or more steps in a particular sequence, it should be understood that such methods may be practiced with the steps in a different order, with certain steps performed simultaneously, with additional steps, and/or with certain described steps omitted.

All matter contained in the above description or shown in the accompanying drawings shall be interpreted as illustrative only and not limiting. Changes in detail or structure may be made without departing from the present disclosure.

It should be understood that an electronic control unit (ECU), a system, and/or a processor as described herein may include a conventional processing apparatus known in the art, which may be capable of executing preprogrammed instructions stored in an associated memory, all performing in accordance with the functionality described herein. To the extent that the methods described herein are embodied in software, the resulting software can be stored in an associated memory and can also constitute means for performing such methods. Such a system or processor may further be of the type having ROM, RAM, RAM and ROM, and/or a combination of non-volatile and volatile memory so that any software may be stored and yet allow storage and processing of dynamically produced data and/or signals.

It should be further understood that an article of manufacture in accordance with this disclosure may include a non-transitory computer-readable storage medium having a computer program encoded thereon for implementing logic and other functionality described herein. The computer program may include code to perform one or more of the methods disclosed herein. Such embodiments may be configured to execute via one or more processors, such as multiple processors that are integrated into a single system or are distributed over and connected together through a communications network, and the communications network may be wired and/or wireless. Code for implementing one or more of the features described in connection with one or more embodiments may, when executed by a processor, cause a plurality of transistors to change from a first state to a second state. A specific pattern of change (e.g., which transistors change state and which transistors do not), may be dictated, at least partially, by the logic and/or code.

What is claimed is:

1. A medical equipment distribution assembly, including:
a housing including one or more medical kits;
an electronic control unit (ECU);
a camera assembly; and
a thermal imaging unit;
wherein the camera assembly and the thermal imaging unit are electrically connected with the ECU such that the ECU may be configured to identify an individual via information received from the camera assembly;
the ECU is configured to determine a medical state of said individual via information received via the thermal imaging unit;
the ECU is configured to determine if said individual requires a medical kit; and
the ECU is configured to distribute the medical kit to said individual via the housing.

2. The medical equipment distribution assembly of claim 1, wherein the camera assembly includes a plurality of cameras wirelessly connected with the ECU.

3. The medical equipment distribution assembly of claim 1, wherein the camera assembly includes a first camera disposed on a first side of the housing.

4. The medical equipment distribution assembly of claim 3, wherein the camera assembly includes a second camera disposed on a second side of the housing.

5. The medical equipment distribution assembly of claim 1, including a mobile assembly; wherein the mobile assembly is connected with the housing such as to move the housing about an area.

6. The medical equipment distribution assembly of claim 5, wherein the ECU is connected with an autonomous server and the autonomous server is configured to at least partially control movement of the housing via the mobile assembly.

7. The medical equipment distribution assembly of claim 6, wherein the autonomous server may be configured to control a plurality of housings.

8. A method of operating an equipment distribution assembly, including:
providing a housing with one or more medical kits, an electronic control unit (ECU), a camera assembly, and a thermal imaging unit;
receiving identifying information of an individual via the camera assembly;
receiving medical information via the thermal imaging unit;
determining if said individual requires a medical kit; and
distributing said medical kit to said individual.

9. The method of claim 8, including determining, via the ECU, whether said individual is wearing appropriate protective equipment.

10. The method of claim 8, wherein distributing includes moving the housing, via a mobile assembly, to a position substantially proximate said individual.

11. The method of claim 8, including sanitizing one or more medical kits prior to distributing via a sanitization portion disposed within the housing.

12. The method of claim 11, wherein the sanitization portion is configured to sanitize one or more medical kits via emitted light.

13. The method of claim 11, wherein the sanitization portion is configured to sanitize one or more medical kits via projected chemicals.

14. The method of claim 8, including sanitizing one or more medical kits after distributing via a sanitization portion disposed proximate the housing.
